(19) United States
(12) Patent Application Publication
(10) Pub. No.: US 2015/0051563 A1
(43) Pub. Date: Feb. 19, 2015

(54) Ostomy stoma waste overflow system

(71) Applicants: Dale Martin Frimel, North Royalton, OH (US); Anita Marie Zindroski, Parma, OH (US)

(72) Inventors: Dale Martin Frimel, North Royalton, OH (US); Anita Marie Zindroski, Parma, OH (US)

(21) Appl. No.: 13/966,634

(22) Filed: Aug. 14, 2013

(51) Int. Cl. A61F 5/445; A61F 5/44

(52) U.S. Cl. CPC A61F 5/445; A61F 5/4405
USPC 604/335

(57) ABSTRACT

An ostomy system and adaptor which allows human waste to flow from one ostomy bag to another ostomy bag via a one directional flow valve which has the ability to automatically open when the stoma waste pressure from the first, or primary, bag reaches a waste pressure near, but under, the pressure that would cause the primary bag to leak onto the person wearing it. Present ostomy bags can leak onto the person wearing it if the bag stoma waste pressure increases by over filling, or by being physically bumped or squeezed (such as when a person is sleeping on it). This new invention prevents an ostomy bag from breaking the ostomy seal (which seals the ostomy body flange to the person's skin) and leaking human waste material onto the person wearing it. Leaking can cause great embarrassment and pain since human waste will burn the skin where the ostomy flange is glued to the user. Leaking can also greatly stain the bed, sheets and blankets where a person is sleeping.

### CROSS REFERENCE TO RELATED APPLICATIONS

| | | | |
|---|---|---|---|
| U.S. PAT. NO. 8,449,511 | Activity Ostomy Bag | Anderson | May 28, 2013 |
| U.S. PAT. NO. 8,211,072 | Ostomy Bag | Smith | Jul. 3, 2012 |
| U.S. PUB. NO. 20130053802 | Ostomy Devices | Maidl | Feb. 28, 2013 |
| U.S. PAT. NO. 8,075,539 | Waste Management | Nishytala | Dec. 13, 2011 |
| | System | | |
| U.S. PAT. NO. 7,918,836 | Ostomy Bag with Irrigation | Gill | Apr. 5, 2011 |
| U.S. PAT. NO. 7,722,586 | Ostomy Appliance | Mallejans | May 25, 2010 |
| U.S. PAT. NO. 7,722,585 | Pouch for Collecting | Falconer | May 25, 2010 |
| | Human Waste | | |
| U.S. PAT. NO. 7,470,263 | Ostomy System | Strobech | Dec. 30 2008 |
| U.S. PAT. NO. 7,416,543 | Drainage Bag | Brown | Aug. 26, 2008 |
| U.S. PAT. NO. 7,223,260 | Collecting Bag | Hansen | May 29, 2007 |
| U.S. PAT. NO. 7,150,728 | Collecting Bag | Hansen | Dec. 19, 2006 |
| | having Vent | | |
| U.S. PAT. NO. 7,001,367 | Valved Ostomy | Arkinstall | Feb. 21, 2006 |
| | Drainage Device | | |
| U.S. PAT. NO. 6,902,551 | Ostomy Appliance | Hansen | Jun. 7, 2005 |
| U.S. PAT. NO. 6,659,988 | Ostomy Appliance | Steer | Dec. 9, 2003 |
| U.S. PAT. NO. 6,656,169 | Ostomy Pouch | Steer | Dec. 2, 2003 |
| U.S. PAT. NO. 6,332,879 | Ostomy Appliance | Nielsen | Dec. 25, 2001 |
| U.S. PAT. NO. 6,165,159 | Gas Vent for Ostomy Bag | Blauton | Dec. 26, 2000 |
| U.S. PAT. NO. 5,658,267 | Colstomy Collection | Cofacello | Aug. 19, 1997 |
| | Pouch | | |

### Classification 604/332

### STATEMENT REGARDING FEDERALLY SPONSORED RESEARCH DEVELOPMENT

Not applicable.

### REFERENCE TO SEQUENCE LISTING, A TABLE, OR A COMPUTER PROGRAM LISTING COMPACT DISK APPENDIX

Not applicable.

### BACKGROUND OF THE INVENTION

A person who has had an ileostomy must use an external bag to collect their human waste material. This waste exits the body from internal organs through an opening in their abdomen called a stoma. There is a latex-like bag that collects human waste material exiting from this stoma. The bag is attached via an ostomy body flange that is held on to the person's abdomen with glue. There is a hole in the center of the flange which surrounds the stoma. There a donut shaped soft gasket that surrounds the stoma and sticks to the skin of the person using it. The present physical structure of this bag is a bag that has two openings. One opening has a plastic, snap-on attachment ring surrounding the opening used to snap onto the body flange mentioned above. The second opening is held closed with a clip until the stoma waste needs to be emptied (when the bag fills or is nearly filled). This second opening allows the waste material to exit the bag. This bag is called an ostomy bag.

The problem with present ostomy collection systems as the one described in paragraph 001 above is that when the ostomy bag becomes full of human waste, there is nowhere for the stoma waste to go when the pressure inside the bag reaches a critical amount. Usually what happens is that the stoma waste is forced out of the bag around the stoma on the person's skin by being forced under the gasket which seals the body flange to the skin surrounding the stoma. When this happens, human waste is spread out onto the skin and clothes of the person wearing it.

Another problem with the present art above is that the ostomy bag can also be bumped or squeezed when a person is sleeping and rolls over on it, or it can be squeezed when the bag is only partially full and bumped against an object such as piece of furniture. When this happens, the stoma waste can be forced out again as described in paragraph 002 above and can soak onto the person's clothes or the bed. This is very embarrassing and unsanitary.

The invention described here prevents the bag from breaking through the stoma ring gasket by allowing the excess stoma waste to be automatically transferred to a second, backup, ostomy bag through a one way valve which opens in the event that the pressure of the stoma waste in the first, primary, ostomy bag reaches a level just under the pressure that would allow the stoma waste to break the body flange seal and spill out on the person wearing the ostomy system.

This invention also has the advantages stated in paragraph 004 above while still making use of present ostomy stoma waste collection units presently on the market, thus making the invention easy to adapt to an ileostomy patient's existing medical supplies, and method of usage.

### BRIEF SUMMARY OF THE INVENTION

This new invention is a device and a method by which stoma waste (from a person who has had an ileostomy) can safely and securely overflow into a second ostomy bag should the first or primary ostomy bag become too full to handle any more intake of stoma waste, or in a situation where the first or primary ostomy bag becomes physically bumped or should happen to be squeezed to such an extent that the stoma waste from the primary bag would otherwise break through the part of the ostomy body flange and onto the person wearing it.

The new invention is an enclosed chamber which can assume any physical shape, but in general have the shape of an oval or rectangle of a thickness that is generally much thinner then it's length or width. It can be made of any construction material and may be flexible or ridged, or a combination of such. It may also be made of a flexible plastic material such that the device is comfortable for a person who has had an ileostomy to wear it. Each person has a different size and shape, and may have a stoma located in such a place as to have one shaped device more comfortable then another. This device stated as the invention may be called an ostomy adaptor, and it's description will prove that out hence forth.

This new adaptor can be fitted with standard plastic ring clips which would make it easy to attach to the various ostomy products now available on the market, which an ileostomy patient would make use of.

The second critical part of the adaptor is a check valve, which is well known and available in various sizes and specifications in the present day market. The purpose of the check valve is to allow stoma waste to overflow into a second, backup, ostomy bag when the stoma waste pressure from the first or primary ostomy bag reaches an amount almost to, but not reaching the pressure that would allow the stoma waste to break through the seal that attaches the ostomy body flange to the skin of the ileostomy patient.

### BRIEF DESCRIPTION OF THE SEVERAL VIEWS OF THE DRAWINGS

FIG. 1 shows three separate views of the ostomy adaptor. The top view shows one side of the adaptor, FIG. 1A, that would connect to two separate, standard, ostomy bags. The primary ostomy bag (the bag that an ileostomy patient would wear today) would connect to an opening in the adaptor shown as FIG. 1B. This primary bag can be snapped onto a connecting ring securing and sealing it to this opening in the adaptor. FIG. 1C shows the outlet opening of the one way check valve, FIG. 1H. FIG. 1D shows an internal chamber which connects two adaptor openings shown in all views of FIG. 1.

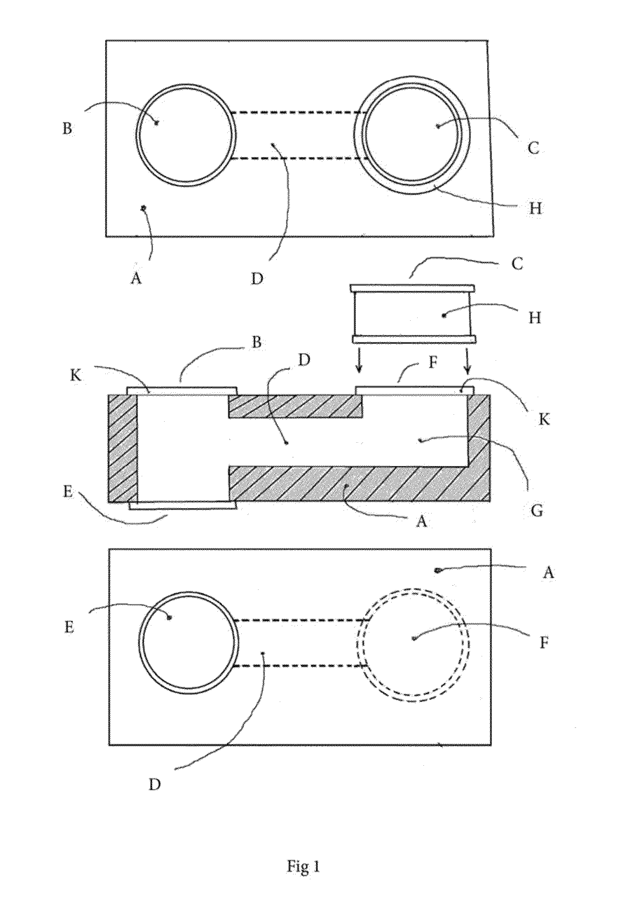

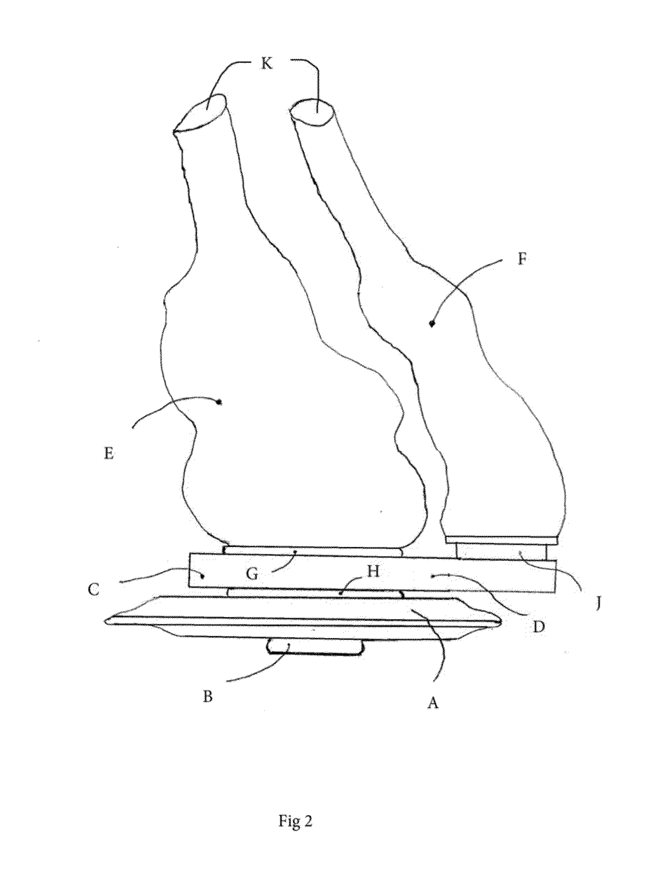

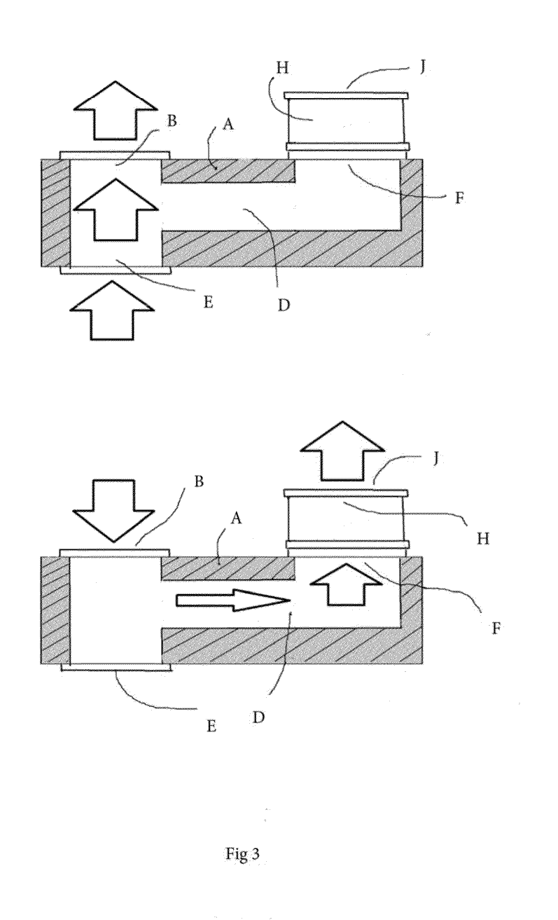

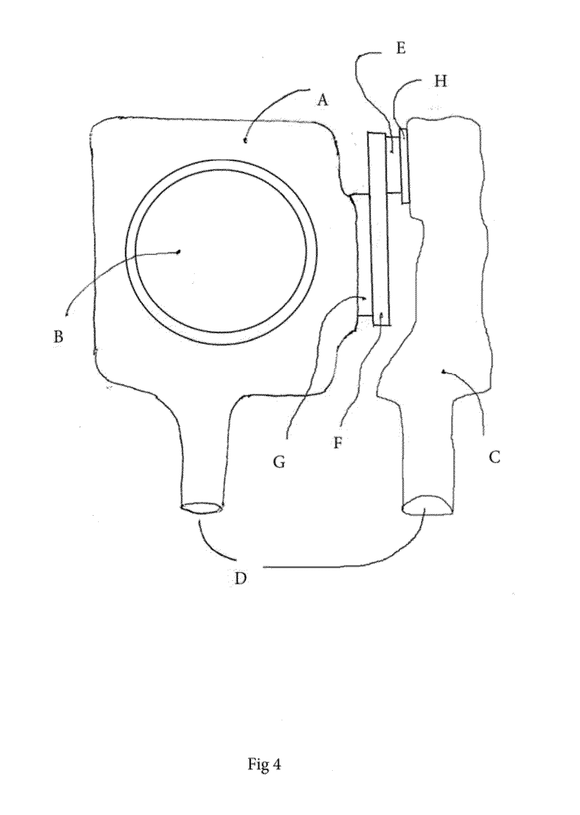

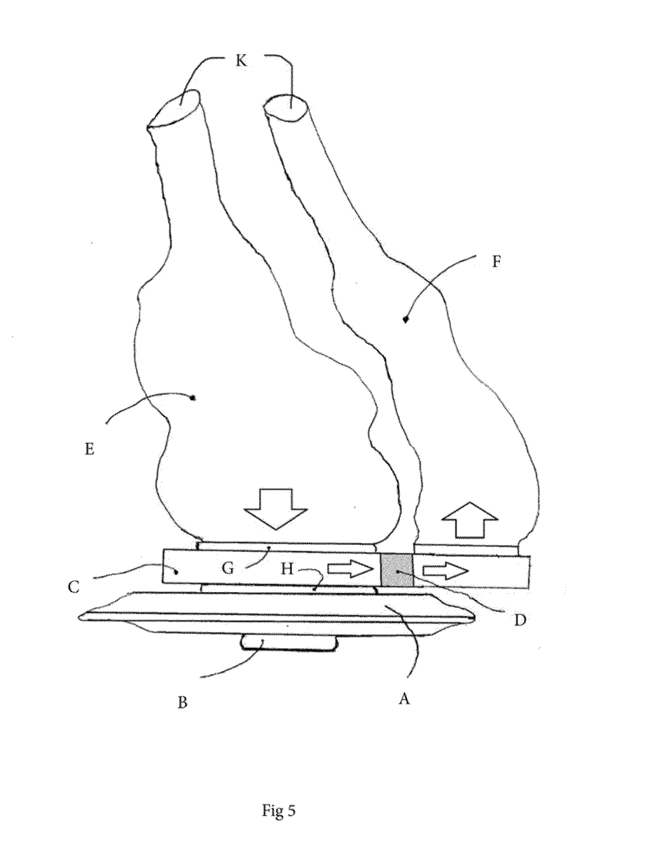

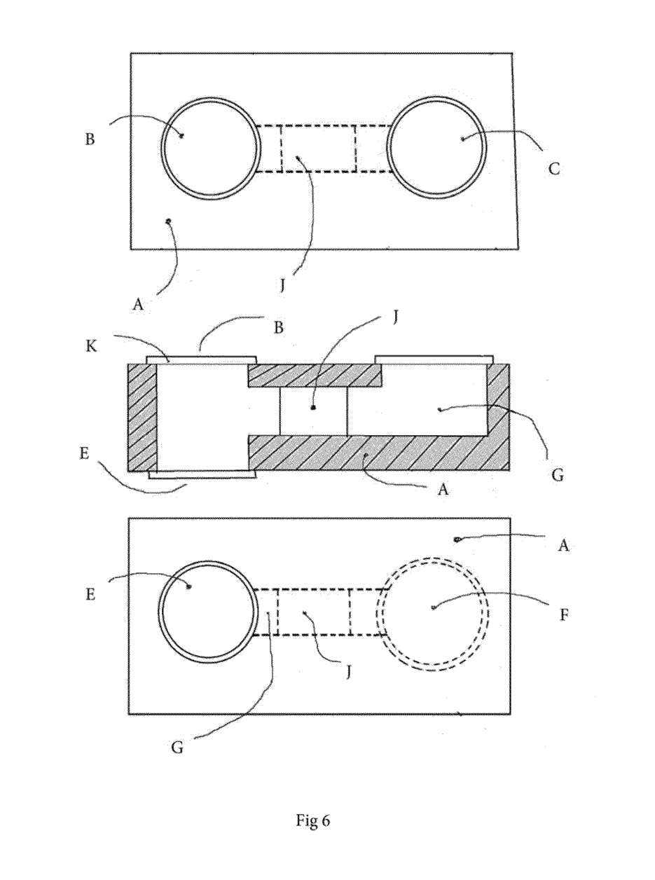

The middle view of FIG. 1, for clarity, shows a sectional view of the adaptor looking at it from the side. FIG. 1B shows the same opening as in FIG. 1B of the top view. FIG. 1D of the middle view shows the internal chamber that connects to all openings in the adaptor, FIGS. 1B, E and F. FIG. 1F is an opening in the adaptor that connects to the inlet side of the one way check valve, FIG. 1H, shown in the middle and the top views. The body of the adaptor is shown in all views as FIG. 1A.

The bottom view in FIG. 1 shows the adaptor body, FIG. 1A, and the internal chamber of the adaptor, FIG. 1D. FIG. 1E in the bottom view of FIG. 1 shows an opening in the adaptor that is open all the way through the adaptor. FIG. 1E, show in the bottom view of the adaptor, would connect directly to an ostomy body flange. This common ostomy body flange is glued to an ileostomy patient and will be shown in subsequent drawings. In the bottom view of FIG. 1, we can see FIG. 1F showing a hidden view of the adaptor's top side opening. This is the same opening as in FIG. 1F of the middle view.

FIG. 2 shows how two ostomy bags would be attached to the ostomy adaptor described thus far. FIG. 2C shows the present invention, the ostomy adaptor. FIG. 2J shows the one way check valve attached between the outlet opening of the adaptor and the inlet of the second, backup, ostomy bag.

FIG. 2A shows a present day ostomy body flange. FIG. 2B shows a present day ostomy body flange gasket. This sticky ring gasket seals the body flange to the patient's skin. It surrounds the patient's stoma. The stoma is where the person's body waste exits the body. FIG. 2E is the primary ostomy bag. FIG. 2F is the secondary, or backup ostomy bag. FIG. 2K shows openings on standard ostomy bags which allow the patient to empty the bags into a common toilet. These end openings are usually clipped shut with a plastic clip while the bags are in use. Usually there is only one bag in use—the primary bag as shown in FIG. 2E. This new invention makes use of two ostomy bags. Both ends of the bags (FIG. 2K) can be clipped closed with one clip. FIG. 2 shows, then, how the various attached units can be used to function as a stoma waste overflow protection system.

FIG. 3 shows cross sectional views of the new ostomy adaptor. The arrows in the top view of FIG. 3 shows the normal direction of stoma waste freely flowing through the adaptor from the body flange side of the adaptor, FIG. 3E, to the primary ostomy bag. The primary ostomy bag stores the stoma waste until it is physically emptied by the user. The top view of FIG. 3 shows the new adaptor, FIG. 3A, the internal chamber, FIG. 3D, the adaptor's overflow outlet opening, FIG. 3F, the one way check valve, FIG. 3H, and the outlet side of the check valve, FIG. 3J. This outlet side of the valve connects to the second ostomy backup bag.

The bottom view of FIG. 3 has arrows that show the direction that the stoma waste would flow through the adaptor, FIG. 3A, in the event that the primary bag becomes filled to a point where the stoma waste pressure opens the check valve. This valve will also open if the stoma waste pressure increases because the primary ostomy bag gets bumped or squeezed. The arrows show here how the excess stoma waste will flow from the primary ostomy bag to the secondary ostomy bag through the adaptor. The waste material will not flow out of the adaptor's opening FIG. 3E onto the person because the pressure of the valve is set at a lower opening pressure then the pressure needed to break through the body seal on the ostomy body flange.

FIG. 4 shows another location where the new adaptor can be connected. Here the primary ostomy bag, FIG. 4A, is connected to the secondary ostomy bag, FIG. 4C through the adaptor, FIG. 4F. The valve is shown in FIG. 4E. FIG. 4H and FIG. 4G are simply plastic connecting rings. These rings are used today with standard ostomy bags and flanges used to connect and seal the units together.

FIG. 5 shows a variation in the use of the new ostomy adaptor. The units are connected together the same as shown in FIG. 2 except that instead of connecting the check valve between the second ostomy bag and the adaptor, the valve is shown integrated within the adaptor's center open channel. The operation is still the same since the excess stoma waste can still flow from the primary ostomy bag to the secondary ostomy bag by flowing through the valve when the overpressure dictates should. The valve is shown here in FIG. 5D. The arrow shows the direction of stoma waste flow when the valve is open. In FIG. 5 the outlet opening of the adaptor can now be connected directly to the second ostomy bag.

FIG. 6 shows the variation described in paragraph 017 above. The valve, FIG. 6J, is shown (in all three views) integrated within the adaptor.

### DETAILED DESCRIPTION OF THE INVENTION

This invention is an adaptor having a container-like shape and volume which can be made of a material which would be comfortable for a person to wear who has had an ileostomy. The adaptor would have two openings on one side with connecting units used for the purpose of securing and sealing two ostomy bags to these openings. The adaptor can have one ostomy bag connected to one opening of the adaptor, and Ban have the inlet side of a check valve connected to the second adaptor opening of the adaptor using the same type of connectors described above and have the outlet end of the check valve connected to the inlet open ng of a second ostomy bag. The purpose of the ostomy adaptor and check valve is to provide a process by which excess stoma waste can flow in the event that the primary waste collecting ostomy bag overfills or is bumped or squeezed. When overfilling or bumping of the ostomy bag occurs, the stoma waste pressure will rise within this closed system. The opening of the one way valve occurs at a preset pressure which would be under the pressure needed to break the ostomy body flange seal. The breaking of this seal (which is the seal between the skin of an ileostomy patient and the ostomy body flange) would cause human waste to be released onto the person and the clothes of the person, or also onto the bed sheets and blankets of the person's bed. This is unsanitary and very distressful for the person wearing such a device. Present art has no way of channeling this overflowing body waste to a secure and sanitary place. When this valve opens, the excess stoma waste can flow to the secondary or backup ostomy bag. When the pressure drops, the valve automatically closes and the waste in the second bag cannot flow back into the first bag. This pressure release gives the user a chance to empty the bags at a later time through the standard present way by taking a plastic clip off of the emptying openings of the bags, and emptying both bags into a common toilet—as the person usually would. Check valves of various dimensions and specifications are common items that, are presently available.

This adaptor would have one opening on the opposite side (opposite the ostomy bag side described in paragraph 019 above) with a similar connector for the purpose of attaching and sealing to a present art ostomy body flange. Normally, only one ostomy bag attaches to a person who needs such a device who has had an ileostomy. There is an opening, in this primary bag which has an device such as a plastic clamping ring, shown in FIG. 2G. This primary bag connects to a flange, FIG. 2A, which is a unit that an ileostomy patient would wear via this connecting ring. The flange has a surrounding area which sticks to the person's skin with glue. The adaptor opening, FIG. 1E would connect to the flange opening, FIG. 2G. Thus the adaptor is sandwiched between the ostomy body flange and two ostomy bags.

FIG. 1 shows the adaptor described above. The opening FIG. 1B goes straight through the adaptor to the opening FIG. 1E. The adaptor opening FIG. 1F is open to the center volume FIG. 1D. This center volume shown in FIG. 1D connects to all the openings in the adaptor.

The one way check valve is shown in FIG. 1H. This check valve attaches to the adaptor opening, FIG. 1F. A present art ostomy connector is used to secure and seal the check valve is shown in FIG. 1K.

FIG. 2 shows how all the units are connected together. FIG. 2A is a present art ostomy body flange. FIG. 2C shows the ostomy adaptor (this invention). FIG. 2J shows the check valve (a part of this invention as it is attached between the ostomy adaptor and a secondary ostomy bag). FIG. 2E is the primary ostomy bag. FIG. 2F shows the secondary ostomy bag.

The purpose of the ostomy adaptor and check valve is to provide a place for excess stoma waste to flow in the event that the primary waste collection ostomy bag overfills or is bumped or squeezed. When overfilling or bumping of the ostomy bag occurs, the stoma waste pressure will rise within this closed system. The opening of the one way valve occurs at a preset pressure which would be under the pressure needed to break the ostomy body flange seal. The breaking of this seal (which is the seal between the skin of an ileostomy patient and the ostomy body flange) would cause human waste to be released onto the person and the clothes of the person, or also to the bed sheets and blankets of the person's bed. This is unsanitary and very distressful for the person wearing such a device. Present art has no way of channeling the body waste to a secure and sanitary place. When this valve opens, the excess stoma waste can flow to the secondary or backup ostomy bag. When the pressure drops, the valve automatically closes and the waste in the second bag cannot flow back into the first bag. This pressure release allows the user time to empty the bags through the standard present way by taking a plastic clip off of the empty openings of the bags, and emptying both bags into a common toilet—as the person usually would, FIG. 3 shows the operation of the invention, and shows how overflow stoma body waste is transferred automatically to a second, backup, ostomy bag. The top view of FIG. 3 shows the normal flow of stoma waste from an ileostomy patient. The arrows in this view shows how the body waste would leave the person through the stoma and flow freely through the adaptor, entering through the opening FIG. 3E, and exiting at FIG. 3B. The top arrows here shows how the waste material flows right into the primary ostomy bag which has the inlet opening of the bag connected to this outlet opening of the adaptor at FIG. 3B. No waste material can flow through the adaptor channel to exit the adaptor's outlet opening at FIG. 3F because at this point the check valve, FIG. 3H is closed. Normally, the primary ostomy bag attaches to a person who needs such a device who has had an ileostomy. There is an opening, in this primary bag which has a device such as a plastic clamping ring, shown in FIG. 2G. This primary bag connects to a body flange, FIG. 2A, which is a unit which an ileostomy patient would wear via this ring. The flange has a surrounding area which sticks to the person's skin with glue.

FIG. 3 shows an ostomy flange adaptor. The top view of FIG. 3 shows the normal flow of stoma waste from a person who has had an ileostomy and wears an ostomy bag. The store a waste inlet side of the primary ostomy bag attaches to FIG. 3B (the adaptor's primary stoma waste outlet opening). The stoma waste inlet side of the backup or secondary ostomy bag connects to FIG. 3F (the adaptor's stoma waste overflow outlet). The adaptor's stoma waste inlet is shown in FIG. 3E. This adaptor opening attaches to the ostomy body flange which is glued to the person. Stoma waste leaves the person and flows freely out of the body flange and through the adaptor through FIG. 3E and FIG. 36 and into the first ostomy bag where it is stored until the bag is emptied by the user. The stoma waste at this point will not flow through the check valve and out of FIG. 3F in to the backup ostomy bag because the primary bag is not filled enough to cause much pressure within this closed system. The check valve only opens when the stoma waste pressure reaches a critical amount.

As the primary bag fills, there may come a point where the bag is full enough, and a situation occurs which causes the primary bag to be physically squeezed from an external force (such as being bumped against an object). When this occurs, there is a spike in internal stoma waste pressure which is enough to open the check valve. The valve opening pressure is less than the pressure needed to break the flange seal on the stomach of the person wearing it. When the valve opens, the stoma waste now can freely flow through the valve, out of the adaptors outlet opening, and into the secondary backup bag. The excess waste is now safe within the second bag. The pressure drops, the valve closes, and now the waste from the secondary bag cannot go back in to the primary ostomy bag. Thus the person wearing the new invention does not experience the hardships of a full ostomy bag leaking on their person. Another advantage of this adaptor is that a user can use standard ostomy bags and a standard ostomy body flange presently sold on the market today.

The adaptor in FIG. 5 performs the same function as in paragraphs above. The difference here is that the check valve, FIG. 50, is integrated within the adaptor while the adaptor's secondary outlet, FIG. 1F, is now attached directly to the inlet opening of the secondary, or backup, ostomy bag.

Stoma waste enters and freely flows through the adaptor by going from the person's stoma into the adaptor's inlet opening. It then moves through the adaptor and out of the adaptor's primary outlet opening, where it empties into the primary ostomy bag, FIG. 5E. As the ostomy bag, FIG. 5E fills, and then overfills, the valve, FIG. 5D, opens under pressure and allows the overflowing waste to pass through the integrated valve, out of the adaptor's outlet opening, and into the secondary backup ostomy bag, FIG. 5F,

FIG. 6 shows the integrated check valve, FIG. 6J, again within the center volume of the ostomy adaptor. FIG. 6 is shown to clarify the location of where the check valve can also be placed.

This new invention provides stoma waste overflow protection to an ileostomy patient by containing the excess overflow of stoma waste in a separate ostomy bag with the use of an ostomy adaptor in conjunction with a check valve.

What is claimed in this invention is:

1. This invention is presented here as an ostomy adaptor which is constructed in the general shape of a closed container with three openings, all common to a central volume within the container. One opening has a connector that can attach to an ostomy bag. A second opening has a connector that can attach to the inlet of a unidirectional check valve. A third opening has a connector that can attach to an ostomy body flange. The outlet of the check valve has a connector that can attach to a second ostomy bag. The purpose of the adaptor is to provide an automatic method of releasing human stoma waste from the first or primary ostomy bag to the secondary or backup ostomy bag through the check valve if the stoma waste pressure in the first or primary ostomy bag becomes too great to be contained within this closed ostomy system.

2. The process by which the adaptor described in claim 1 above can release human stoma waste, and route the excess waste material to a second ostomy bag from the primary or first ostomy bag when the primary bag becomes too full to collect any more waste material, or becomes bumped so as to cause the pressure in the first ostomy bag to rise to a point just below the pressure that would cause the waste material to break through an ostomy flange and seal, and onto the person wearing an ostomy device.

3. The adaptor as described in claim 1 above with the exception that the check valve is integrated within the adaptor, located within the central volume of the adaptor, rather than having the check valve connected to one of the outlet Openings of the adaptor. The function and operation of the adaptor would be exactly the same as in claim 1.
